(19) United States
(12) Patent Application Publication
(10) Pub. No.: US 2011/0282064 A1
(43) Pub. Date: Nov. 17, 2011

(54) METHOD FOR THE MANUFACTURE OF HIGHLY PURE PRASUGREL

(76) Inventors: Hana Stepankova, Cesky Brod (CZ); Josef Hajicek, Praha (CZ); Michal Dousa, Klatovy (CZ)

(21) Appl. No.: 13/129,727

(22) PCT Filed: Nov. 24, 2009
(86) PCT No.: PCT/CZ09/00139

§ 371 Date: Aug. 1, 2011

(30) Foreign Application Priority Data
Nov. 26, 2008 (CZ) PV 2008-748

(51) Int. Cl. C07D 495/04

(52) U.S. Cl. 546/114

(57) ABSTRACT

The invention deals with preparation of the substance prasugrel, using 3-cyclopropyl-1-(2-fluorophenyl)-3-oxopropyl methanesulfonate for alkylation of 2-oxo-thienotetrahydropyridine, which may be in the form of a salt, e.g. with hydrochloric acid or p-toluenesulfonic acid. The resulting compound of formula II is acylated, preferably with acetanhydride, preferably directly in the reaction mixture without isolation, and the produced prasugrel of formula I can then be crystallized directly from the reaction mixture.

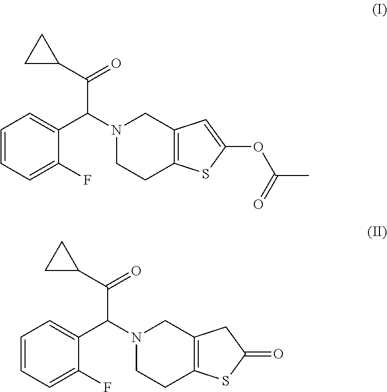

### TECHNICAL FIELD

The invention deals with a new method of manufacturing 5-[2-cyclopropyl-1-(2-fluorophenyl)-2-oxoethyl]-4,5,6,7-tetrahydrothieno[3,2-c]pyridin-2-yl acetate, known under the non-proprietary name prasugrel, in high purity. Prasugrel is a well-known substance reducing blood coagulation, of formula I

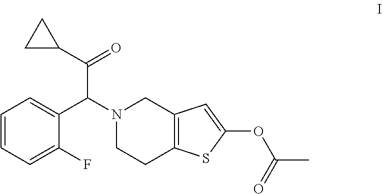

### BACKGROUND ART

Prasugrel, a method of its preparation and its use as an anti-aggregation substance for patients with the risk of blood vessel obstruction by a blood clot was first described in the patent no. EP 542411.

Manufacture of prasugrel in accordance with this patent can be summarized in Scheme 1.

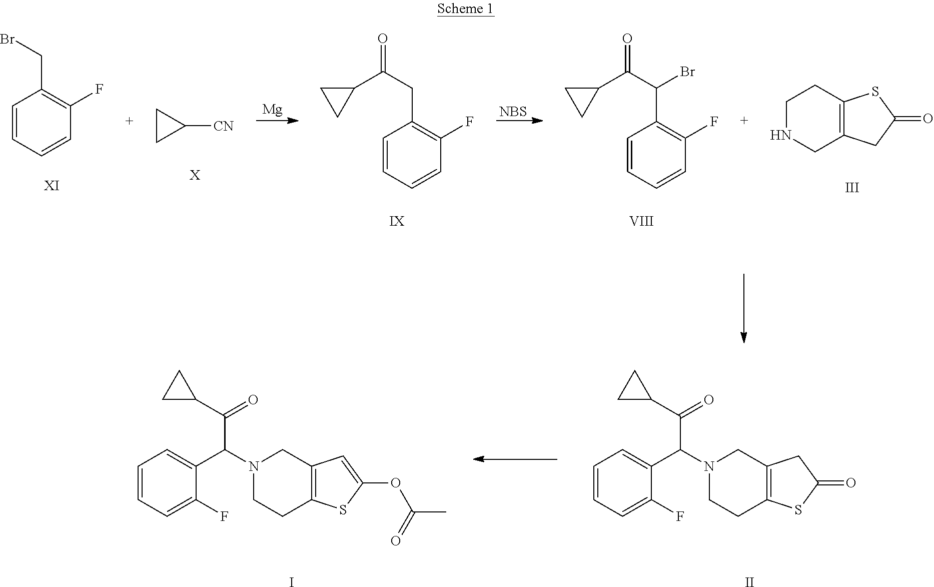

According to this document the Grignard reagent prepared from 2-fluorobenzylbromide (XI) reacts in ether with cyclopropylcyanide (X) and provides the compound (IX). The compound (IX) reacts with bromine in CCl₄ or with N-bromosuccinimide (NBS) in the presence of dibenzoylperoxide to the bromine derivative (VIII), which is added in the presence of potash to the nitrogen atom of the compound (III), producing the compound (II). The compound (II) is transformed to the final prasugrel (I) by reaction with acetanhydride in the presence of NaH in DMF.

A similar method can be deduced from an older document no. EP 192 535, which is outlined in Scheme 2.

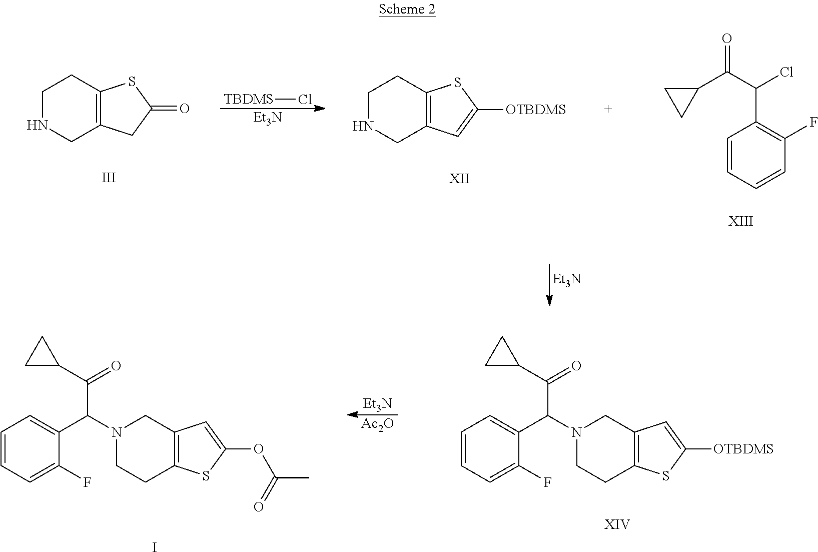

A reaction of thienopyridin-2-one (III) with tert-butyldimethylsilylchloride (TBDMS-Cl) in dichloromethane in the presence of triethylamine provides silylated enolether (XII), which reacts with the compound (XIII), again in the presence of triethylamine in dichloromethane, to the compound (XIV). The final prasugrel of formula I is then prepared from the substance (XIV), first after additional protection with Et₃N and subsequent acetylation with acetanhydride in the presence of dimethylaminopyridine.

Besides α-haloketones (VIII) and (XIII) another key intermediate is 2-oxo-thienotetrahydropyridine (III), which is used in the hydrochloride form in Scheme 1 and in the tosylate form in Scheme 2. Its preparation has been described by the Sanofi Company and starts from the commercially available 4,5,6,7-tetrahydrothieno[3,2-c]pyridine (XX); see Scheme 3.

First, the nitrogen atom is blocked by reaction of triphenylmethylchloride in dichloromethane in the presence of Et₃N (96%) and the protected compound (XIX) is prepared. This compound (XIX) is converted to the lithium salt (XVIII), which provides, by reaction with tri-n-butylborate, the derivative (XVII), which is oxidized in-situ with 30% hydrogen peroxide to the compound (XVI), which is immediately hydrolyzed to trityled thienopyridone (XV) (64%). This reaction step is carried out in a mixture of THF and hexane at temperatures of −40° C. to −20° C. In the last step the trityl group is deprotected with 98% formic acid (90° C., 1 hour) (81%) and the desired compound (III) is obtained.

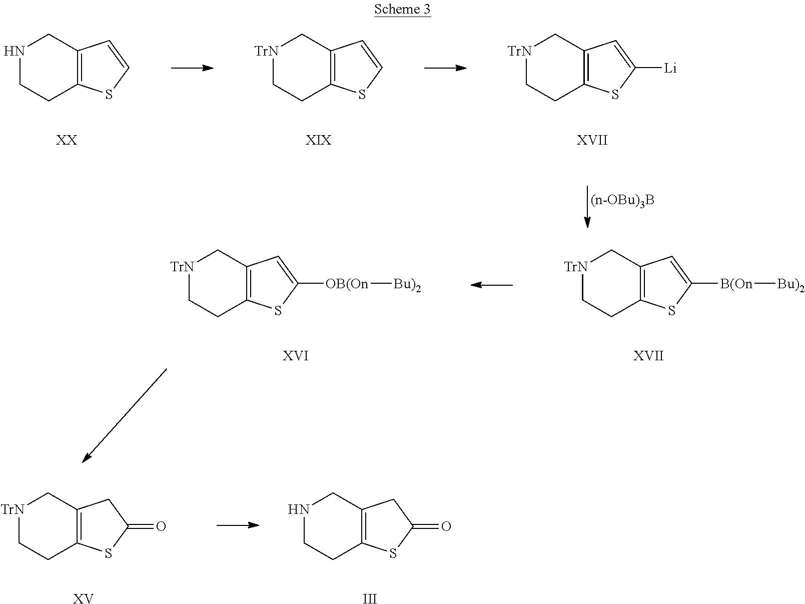

In comparison to the known methods the production method in accordance with the invention offers a technologically feasible preparation procedure that provides the prasugrel base in a high purity. It uses a simple approach without the necessity to use protective groups.

The prasugrel base of formula I is an instable compound; it changes into the compound of formula II according to Scheme 5 under heat load, e.g. during crystallization, only due to its presence in a solution.

Obtaining highly pure prasugrel of formula I is the basic precondition for applicability of a preparation method in the industrial scale.

### DISCLOSURE OF INVENTION

The invention provides a new manufacturing method of highly pure 5-[2-cyclopropyl-1-(2-fluorophenyl)-2-oxoethyl]-4,5,6,7-tetrahydrothieno[3,2-c]pyridin-2-yl acetate, known under the non-proprietary name prasugrel, of formula I.

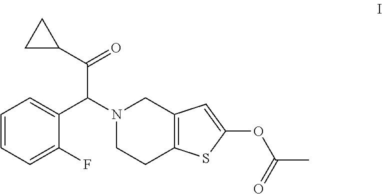

The starting substance of formula IV

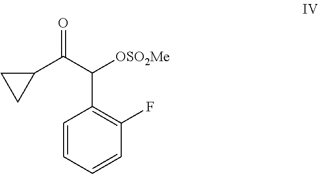

is reacted with the compound of formula III

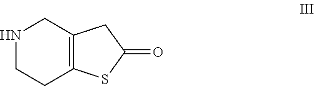

in the form of a salt such as hydrochloride or p-toluenesulfonate, to give the substance of formula II,

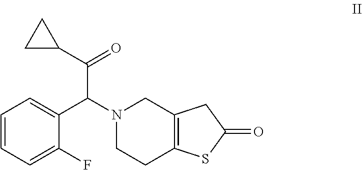

which is then transformed, without isolation, to the substance of formula I using an acetylating agent.

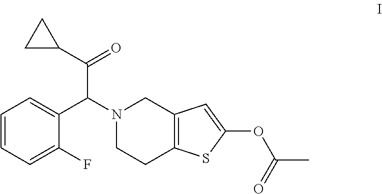

The invention also provides crystallization purification of the product of formula I obtained this way in a solvent with an addition of acetanhydride. Nitriles of organic acids, ethers and cyclic ethers can be used as the solvents. After addition of water or an aqueous solution of an inorganic salt to this mixture prasugrel of formula I is obtained in a high purity with the content of compound of formula II up to 0.2%.

### DETAILED DESCRIPTION OF THE INVENTION

The synthesis according to the invention can be briefly described by the following Scheme 4.

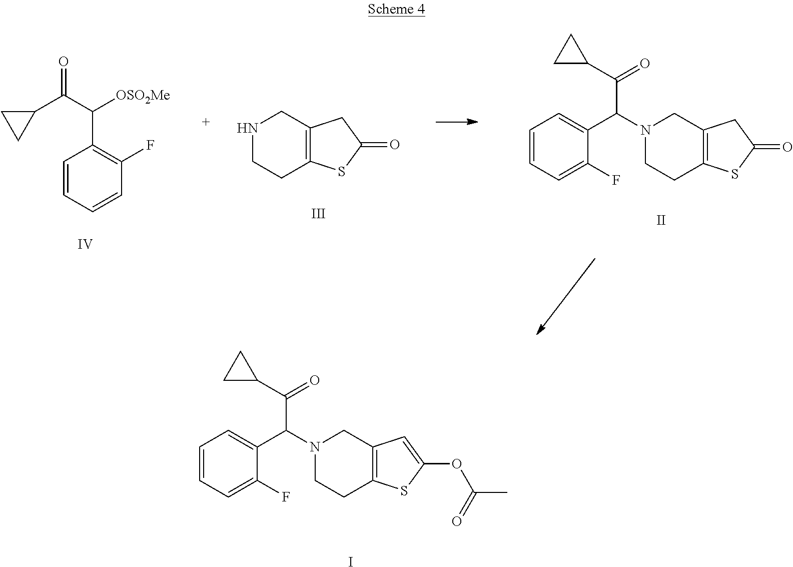

The invention relates to the preparation of the substance prasugrel by a method using 3-cyclopropyl-1-(2-fluorophenyl)-3-oxopropyl methanesulfonate (IV) for alkylation of 2-oxo-thienotetrahydropyridine (III), which may be in the form of a salt, e.g. with hydrochloric or p-toluenesulfonic acid. The resulting compound of formula II is then acylated with an acylation agent directly in the reaction mixture without isolation and the produced prasugrel of formula I is then crystallized directly from the reaction mixture. Acetanhydride or acetylchloride, e.g., are used as the acylation agents. Acetanhydride appears to be the most convenient one.

Prasugrel is an instable compound; it changes into the compound of formula II according to Scheme 5 under heat load, e.g. during crystallization, only due to its presence in a solution.

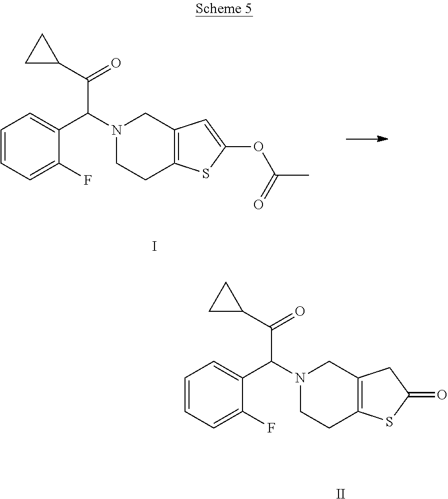

The invention also relates to crystallization purification of the obtained product of formula I in a solvent with the addition of an acylation agent, e.g. acetanhydride or acetylchloride. Acetanhydride appears to be the most convenient one. Polar aprotic solvents are used as the solvents, e.g. nitriles of organic acids, ethers and cyclic ethers. The process is preferably carried out at temperatures of −20 to +50° C. After adding of water or an aqueous solution of an inorganic salt (e.g. a solution of potassium hydrogen phosphate) to this mixture prasugrel of formula I is obtained in high purity. The addition of the acylation agent shifts the equilibrium towards the desired product (I) and prevents from formation of the undesired product of deacylation. The content of the undesired compound of formula II is then lower than 0.2%, preferably lower than 0.1%, which is a purity degree that is acceptable for a pharmaceutical substance. This purification method certainly represents a great technological benefit as the previous methods have always provided the product with a substantially higher content of impurities, especially the deacetylation product of formula II, which was often higher than 3.4%. Attempts to use different reaction conditions, e.g. different temperatures and reaction times as well as attempts with different solvents for the reaction and crystallization did not lead to satisfactory results either, as documented especially by examples nos. 9, 10 and 11.

### EXAMPLES

The purity of prasugrel in the examples mentioned below was evaluated by means of HPLC chromatography using the method as shown in example 6.

### Example 1

Into a 250-ml three-neck flask equipped with a magnetic stirrer and a thermometer, which is closed with a calcium chloride tube, 12.22 g of p-toluenesulfonate of the compound of formula III and 40 ml of acetonitrile are charged. Under stirring, 13.6 ml of diisopropylethylamine are poured to the thick suspension and the mixture is stirred at the room temperature until a solution is obtained (5-10 minutes). Then, 3-cyclopropyl-1-(2-fluorophenyl)-3-oxopropyl methanesulfonate (compound of formula IV) (9.68 g) and 7.84 g of Et₄N⁺Br are added to the flask. After that, the resulting mixture is stirred at a temperature of +22 to +25° C. for 4 to 5 hours. The reaction is monitored with TLC. After disappearance of the starting substance 10 ml of Ac₂O and 50 mg of dimethylaminopyridine are added to the reaction mixture. The reaction mixture is further stirred at a temperature of +22 to +25° C. for another 1.5 to 2 hours. The reaction is monitored with TLC in the same system. After the conversion of the intermediate (II) the reaction mixture is cooled down to a temperature of −12 to −15° C., 25 ml of a 20 mM aqueous solution of KH₂PO₄ are added. The mixture is inoculated and the product is left to crystallize under stirring at a temperature of −12 to −15° C. for 1.5 hours. The separated product is aspirated through a frit and on the frit it is washed with 20 ml of cooled ethanol. The product is freely dried at the room temperature. 4.06 g of the crude product are obtained with the purity of 96.11% (HPLC).

¹H NMR (250 MHz, CDCl₃) δ(ppm): 7.47 (ddd, J=14.7, 7.4, 1.7 Hz, 1H), 7.31 (m, 1H), 7.14 (m, 2H), 6.26 (s, 1H), 4.82 (s, 1H), 3.51 (m, 2H), 2.89 (m, 1H), 2.79 (m, 3H), 4.30 (m, 1H), 2.25 (s, 3H), 1.03 (m, 2H), 0.85 (m, 2H); ¹³C NMR (250 MHz, CDCl₃) δ(ppm): 207.7, 167.7, 161.3 (d, J_{CF}=247.6 Hz), 149.5, 130.6 (d, J_{CF}=3.5 Hz), 129.9 (d, J_{CF}=8.4 Hz), 129.4, 125.8, 124.4 (d, J_{CF}=3.5 Hz), 122.1 (d, J_{CF}=14.1 Hz), 115.8 (d, J_{CF}=22.9 Hz), 112.0, 71.6, 50.5, 48.4, 25.0, 20.6, 18.3, 12.0, 11.4.

### Example 2

Prasugrel prepared in accordance with example 1 (4.06 g) is dissolved in 60 ml of acetonitrile at the room temperature. 2 ml of Ac₂O are added to the solution and the solution is stirred at a temperature of +22 to +25° C. for 1.5 hours. Then, the solution is cooled down to a temperature of −12 to −15° C., and 30 ml of a 20 mM aqueous solution of KH₂PO₄ are added. Under stirring the product is left to crystallize at a temperature of −12 to −15° C. for 2.5 hours. The separated product is aspirated through a frit and washed with a mixture of acetonitrile:water; 1:1. The product is freely dried in the air until a constant weigh is achieved −3.19 g of purified prasugrel are obtained (78.6%); HPLC 99.5%; compound of formula II: 0.07%.

### Example 3

Prasugrel prepared in accordance with example 1 (0.8 g) is dissolved in 11.8 ml of acetonitrile at the room temperature. 1 ml of Ac₂O is added to the solution and the solution is stirred at the room temperature for 10 minutes. The solution is then cooled down to a temperature of −10 to −15° C., and 6.5 ml of a 20 mM aqueous solution of KH₂PO₄ are added. The product is left to crystallize under stirring at a temperature of −12 to −15° C. for 1.5 hours. The separated product is aspirated through a frit and washed with a mixture of acetonitrile:water; 1:1. The product is freely dried in the air until a constant weigh is achieved −0.55 g of purified prasugrel are obtained (68.75%); HPLC 99.11%; compound of formula II: 0.60%.

### Example 4

Prasugrel prepared in accordance with example 1 (0.373 g) is dissolved in 5.5 ml of acetonitrile at the room temperature. The clear solution is cooled down to the temperature of −5° C. 3 ml of a 20 mM aqueous solution of KH₂PO₄ are added to the solution and the product is crystallized at this temperature for 1.5 hours. The separated fraction is aspirated through a frit and washed with a minimum quantity of the mixture of acetonitrile:water; 1:1. 310.7 mg (83.3%) of purified prasugrel are obtained with the content of 98.07%; compound of formula II: 1.7%.

### Example 5

Prasugrel prepared in accordance with example 1 (0.373 g) is dissolved in 3.0 ml of acetone at the room temperature. The clear solution is cooled down to the temperature of −3° C. 1 ml of a 20 mM aqueous solution of KH₂PO₄ is added to the solution and the product is crystallized at a temperature of −5 to 0° C. for 1.5 hours. The separated fraction is aspirated through a frit and washed with a minimum quantity of the mixture of acetonitrile:water; 1:1. 336 mg (90.1%) of purified prasugrel are obtained with the content of 98,127%; compound of formula II: 1.61%.

### Example 6

HPLC determination is carried out in an octadecyl column (250×4.6 mm; 5 μm) at the temperature of 30° C. with UV detection at 228 nm. For the separation gradient elution with a phosphate buffer (0.01 M KH₂PO₄ pH 2.2) with acetonitrile is used at the flow rate of 1.0 ml/min with the following gradient: 0 min 80% of the buffer; 40 min 10% of the buffer (linear gradient); 45 min 10% of the buffer. The equilibration time of the column is 10 minutes. The injection volume is 10 μl. The capacity factor of prasugrel is 4.3. The sample is prepared by dissolution of the corresponding substance in acetonitrile up to the concentration of 1 mg/ml.

### Example 7

Prasugrel prepared in accordance with example 1 (200 mg) is dissolved in 2 ml of tetrahydrofuran at the room temperature. 0.25 ml of Ac₂O are added to the solution and the solution is stirred at a temperature of +22 to +25° C. for 2 hours. The solution is then cooled down to a temperature of −5 to −2° C.; 1 ml of a 20 mM aqueous solution of KH₂PO₄ is added. The product is left to crystallize under stirring at a temperature of −5 to −2° C. for 2.0 hours. The separated product is aspirated through a frit and washed with the solution of THF:water; 1:1. The product is freely dried in the air until a constant weight is achieved −75 mg of purified prasugrel are obtained with the content of 99.45%.

### Example 8

Prasugrel prepared in accordance with example 1 (200 mg) is dissolved in 2 ml of 1,4-dioxan at the room temperature. 0.25 ml of Ac₂O are added to the solution and the solution is stirred at a temperature of +22 to +25° C. for 2 hours. The solution is then cooled down to a temperature of −5 to −2° C.; 1 ml of a 20 mM aqueous solution of KH₂PO₄ is added. The product is left to crystallize under stirring at a temperature of −5 to −2° C. for 2.0 hours. The separated product is aspirated through a frit and washed with the solution of dioxan:water; 1:1. The product is freely dried in the air until a constant weight is achieved −142 mg of purified prasugrel are obtained with the content of 99.80%.

### Example 9

Prasugrel prepared in accordance with example 1 (1.56 g) is dissolved, under stirring and at a temperature of 60° C., in 22 ml of methanol with the addition of an aqueous solution of KH₂PO₄ in the proportion of 20 ml of methanol and 0.5 ml of this solution. After dissolution the heating is immediately turned off and during 0.5 hours the temperature is left to cool down to the room temperature. Crystals start to be separated. The resulting mixture is cooled in a water+ice bath still for 1 hour. The separated product is aspirated and washed with methanol. The product is dried freely in the air until a constant weight is achieved −1.25 g of purified prasugrel are obtained with the content of 97.65%; compound of formula II: 1.48%.

### Example 10

Prasugrel prepared in accordance with example 1 (373 mg) is dissolved in 3 ml of acetone at a room temperature. Under stirring the solution is cooled down to −3° C. and 1 ml of a 20 mM solution of KH₂PO₄ is added. The product is left to crystallize at a bath temperature of −5° C. to 0° C. The separated product is aspirated through a frit and washed with acetone. The product is freely dried in the air until a constant weight is achieved −336 mg of purified prasugrel are obtained with the content of 98.12%; compound of formula II: 1.64%.

### Example 11

Prasugrel prepared in accordance with example 1 (204 mg) is dissolved in 2 ml of acetone at the room temperature. Under stirring the solution is cooled down to −5° C. and 2 ml of methanol are added. The product is left to crystallize at a bath temperature of −5° C. to −10° C., then at −22° C. for 1 hour. The separated product is filtered through a frit and washed with acetone. The product is freely dried in the air until a constant temperature is achieved −96.2 mg of purified prasugrel are obtained with the content of 96.34%; compound of formula II: 3.42%.

1. A method for the preparation of crystalline prasugrel of formula I 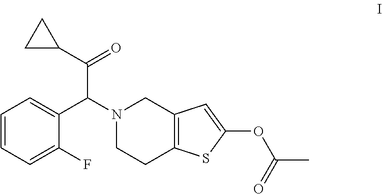
wherein prasugrel is crystallized from aprotic polar solvents in a mixture with water or aqueous solutions, in the presence of an acetylation agent.

2. The method according to claim 1, wherein prasugrel is prepared in an aprotic polar solvent by acetylation of the substance of formula II 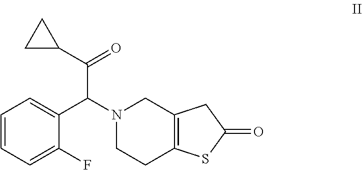
with an excess of the acetylation agent, followed by addition of water or an aqueous solution of an inorganic salt to the reaction mixture.

3. A method of manufacturing highly pure prasugrel, chemically 5-[2-cyclopropyl-1-(2-fluorophenyl)-2-oxoethyl]-4,5,6,7-tetrahydrothieno[3,2-c]pyridin-2-yl acetate of formula I 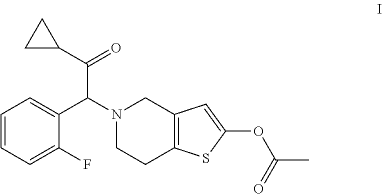
wherein the compound set forth in formula IV 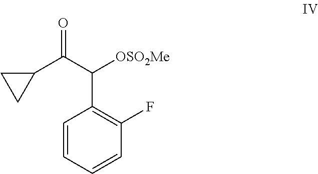
is reacted with the a compound as set forth in formula III 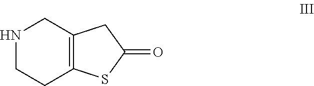
in the form of a salt with hydrochloric acid or with p-toluenesulfonic acid, thereby producing the compound set forth in formula II, 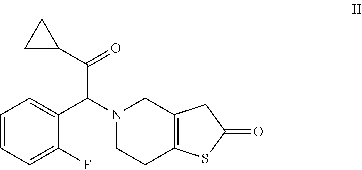
which is then transformed to the compound of formula I with an acetylation agent directly in the reaction mixture and without isolation.

4. The method according to claim 2, wherein the compound of formula II is acylated with acetanhydride directly in the reaction mixture without isolation of the intermediate II and the resulting prasugrel of formula I is then crystallized directly from the reaction mixture.

5. The method according to claim 1, wherein the product I is subsequently re-purified by crystallization in an organic solvent with the addition of an acetylation agent.

6. The method according to claim 5, wherein crude prasugrel is dissolved in a polar aprotic solvent at a temperature of 10 to 50° C., an acetylation agent is added to the solution and subsequently prasugrel crystallizes by the action addition of water or an aqueous solution.

7. The method according to claim 6, wherein product I is re-purified by crystallization in an organic solvent, selected from nitriles of organic acids, ethers and cyclic ethers.

8. A method for the preparation of highly pure prasugrel according to claim 1, wherein product I is re-purified by crystallization in an organic solvent and an addition of an aqueous solution of potassium hydrogen phosphate is used in the isolation process.

9. Prasugrel, chemically 5-[2-cyclopropyl-1-(2-fluorophenyl)-2-oxoethyl]-4,5,6,7-tetrahydrothieno[3,2-c]pyridin-2-yl acetate with high purity, containing not more than 0.2% of 5-[2-cyclopropyl-1-(2-fluorophenyl-2-oxoethyl]-2-oxo-4,5,6,7-tetrahydrothieno[3,2-c]pyridine of formula II.

10. The Prasugrel according to claim 9, containing not more than 0.1% of 5-[2-cyclopropyl-1-(2-fluorophenyl)-2-oxoethyl]-2-oxo-4,5,6,7-tetrahydrothieno[3,2-c]pyridine of formula II.
